(19) United States
(12) Patent Application Publication
(10) Pub. No.: US 2026/0338897 A1
(43) Pub. Date: Oct. 29, 2026

(54) SYSTEM AND METHOD FOR PREPROCESSING OF IMAGES

(71) Applicant: Springbok, Inc., Charlottesville, VA (US)

(72) Inventors: Xue Feng, Charlottesville, VA (US); Lara Riem, Charlottesville, VA (US); Silvia Blemker, Charlottesville, VA (US)

(21) Appl. No.: 19/196,626

(22) Filed: May 1, 2025

Related U.S. Application Data
(60) Provisional application No. 63/794,698, filed on Apr. 25, 2025.

(51) Int. Cl. G01R 33/56; A61B 5/055

(52) U.S. Cl. CPC G01R 33/5608; A61B 5/055; G01R 33/5602; G01R 33/5607; A61B 2576/00

(57) ABSTRACT

The present disclosure provides a system and method for performing a preprocessing workflow on magnetic resonance (MR) image data acquired from a patient at different scan instances, on different MR imaging devices, acquired by different technicians, when the patient has moved with a scan, or any combination thereof. The system and method described herein enable flexibility to process DICOM file data outside of strict protocol compliance to form a full 3D model of the patient using MR image data from various MR image data sources.

### CROSS-REFERENCE TO RELATED APPLICATIONS

This application claims priority to U.S. Provisional Patent Application No. 63/794,698, filed Apr. 25, 2025, and titled SYSTEM AND METHOD FOR PREPROCESSING OF IMAGES, the entirety of which is incorporated herein by reference.

### BACKGROUND

Most magnetic resonance imaging (MRI) scans use small fields of view (FOV) to focus on imaging specific organs, such as the brain, heart, spinal cord, knee, and so forth. Scans are acquired and saved in a Digital Imaging and Communications in Medicine (DICOM) file format as meta data. This meta data provides information regarding acquisition, such as, but not limited to, scanner type, patient-specific identifiers, resolution, and imaging type (e.g., T2, T1, water or fat phase, etc.) of the image acquired.

One problem with DICOM file formats is that the MRI and meta data's tagging systems can vary greatly between MRI manufacturer, model, software version, imaging site, administrating technician, and scanning protocol. Many manufacturers set rigid protocols where failure to accurately accomplish a single step could mean an entire MRI scanning sequence could be marked as “unusable”-requiring further inefficient manual processing to include the otherwise acceptable imaging data. For example, some vendors require technicians to specifically name each series according to the body region captured (“Legs”, “Left Arm”, “Torso”, etc.) and each region must be of a consistent, set resolution/matrix. If the technician forgets to name the series or sets an incorrect resolution, the data cannot be used. Therefore, the failure rate of the AMRA scan is very high. Furthermore, this rigidity also disables the company from using retrospective data, in which the images capture the same regions outside their protocol.

Another common issue is when a patient moves during the scan of a region and that movement is not recorded by the imaging technician. For an example, a patient's left arm may be captured when the patient leans left on the scanner table to have the entire left arm in the FOV, then the patient is moved to the right in order to include the right arm in the FOV. If the patient movement is not recorded when this data is read in the inferior superior location of the acquired images, it might show image overlap as the scanner goes from pelvis to neck on the left and right arm of the patient because of the change in position and failure to update the refence of the patient position. Therefore, attempting to combine images will create an erroneous combination of the left and right side of the patient merged together.

Thus, systems and methods are needed to avoid depending on imaging technicians to have properly denoted specific events in the meta data and allow flexible naming conventions to be able to process large amounts of data from numerous sources.

### SUMMARY OF THE DISCLOSURE

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention. Like reference numerals will be used to refer to like parts from Figure to Figure in the following description.

The systems and methods described herein address the aforementioned drawbacks by providing DICOM preprocessing to 1) separate like images taken from the same patient region (i.e. same location reference), 2) separate like images of similar imaging type, 3) sort the like images between each other based on image location and 4) combine the like images to create one or more continuous 3D volumes by unifying their respective changes in acquisition resolution, FOV, matrix, etc.

In one aspect, the present disclosure provides a system for preprocessing magnetic resonance imaging (MRI) scans of a subject acquired with a medical imaging system. The system comprises a memory configured to store one or more MRI scans of a subject, wherein each MRI scan includes one or more Imaging and Communications in Medicine (DICOM) files acquired from a single study instance unique identifier (UID), and wherein each DICOM file includes a two-dimensional (2D) image array slice and a processor. The processor is configured to access the one or more MRI scans, separate the one or more DICOM files grouped based on one or more series instance UIDs and regroup the one or more DICOM files into one or more series, identify and record one or more properties related to the one or more series and group the series based on one or more proprieties into different series types; sort the slices from the regrouped DICOM files in order of each slice's location along an image patient position of the subject, merge the sorted slices from each series type into a single three-dimensional (3D) image array and record a position value for each of the slices in the 3D image array, form a list of one or more groups comprising the 3D image arrays that are combinable based on sharing the same series type and a region of the subject, transform one or more properties of the one or more 3D image arrays of each series type in each group to be equal, and combine the one or more transformed 3D image arrays of each group using the position value of each slice of the 3D image arrays and a slice spacing.

In another aspect, the present disclosure provides a method for preprocessing magnetic resonance imaging (MRI) scans of a subject acquired with a medical imaging system. The method comprises accessing the one or more MRI scans, wherein each MRI scan includes one or more Imaging and Communications in Medicine (DICOM) files acquired from a single study instance unique identifier (UID), and wherein each DICOM file includes a two-dimensional (2D) image array slice, separating the one or more DICOM files grouped based on one or more series instance UIDs and regrouping the one or more DICOM files into one or more series, identifying and recording one or more properties related to the one or more series and group the series based on one or more proprieties into different series types; sorting the slices from the regrouped DICOM files in order of each slice's location along an image patient position of the subject, merging the sorted slices from each series type into a single three-dimensional (3D) image array and recording a position value for each of the slices in the 3D image array, forming a list of one or more groups comprising the 3D image arrays that are combinable based on sharing the same series type and a region of the subject, transforming one or more properties of the one or more 3D image arrays of each series type in each group to be equal, and combining the one or more transformed 3D image arrays of each group using the position value of each slice of the 3D image arrays and a slice spacing.

### BRIEF DESCRIPTION OF THE DRAWINGS

FIG. 1 is a block diagram of a non-limiting example MRI system that can implement the methods described in accordance with the present disclosure.

FIG. 2A is a flowchart of non-limiting example steps for a method to provide MRI image preprocessing.

FIG. 2B is a flowchart of non-limiting example of detailed steps of identifying series properties within DICOM files from the flowchart of FIG. 2A.

FIG. 2C is a flowchart of non-limiting example of detailed steps of sorting 2D image array slices from the flowchart of FIG. 2A

FIG. 2D is a flowchart of non-limiting example of detailed steps of grouping 3D image arrays and transforming them for uniformity from the flowchart of FIG. 2A

FIG. 2E is a flowchart of non-limiting example of detailed steps of combining 3D image arrays to form a multi FOV image from the flowchart of FIG. 2A

FIG. 3 is a flowchart of non-limiting example steps for a method to correct for component swapping of the method of FIGS. 2A-2E.

FIG. 4A is a non-limiting example of performing the general flowchart of FIG. 2A.

FIG. 4B-4C are non-limiting examples of performing steps in the flowchart of FIGS. 2B-2C.

FIG. 4D is a non-limiting example of the steps performed in FIG. 2D

FIG. 4E is a non-limiting example of the details performed in FIG. 2D and FIG. 4D.

FIG. 4F is a non-limiting example of the steps performed in FIG. 2E.

FIG. 4G is a non-limiting example of the component swapping flowchart of FIG. 3.

FIG. 5 is a block diagram of a system for preprocessing image data in accordance with some implementations of the disclosed subject matter.

FIG. 6 is a block diagram showing an example of hardware that can be used to implement the system of FIG. 5 in accordance with some implementations of the disclosed subject matter.

### DETAILED DESCRIPTION

The present disclosure provides systems and methods to preprocess medical images, such as MRI image, acquired at adjacent fields of view (FOVs) to create images and analysis of an extended view of the musculoskeletal system. Providing extended images and analysis can include generating a report for a desired contiguous volume of interest that is larger than a single acquired FOV. As non-limiting examples, such preprocessing enables the use of medical images acquired at different times, from different imaging systems, and different imaging technicians. Thus, flexibility is afforded to disparate imaging instances of a patient that would otherwise require strict adherence to vendor-specific protocols.

The process can be fully automated to circumvent the need for error-prone and time-consuming manual input, providing standardized and fast results.

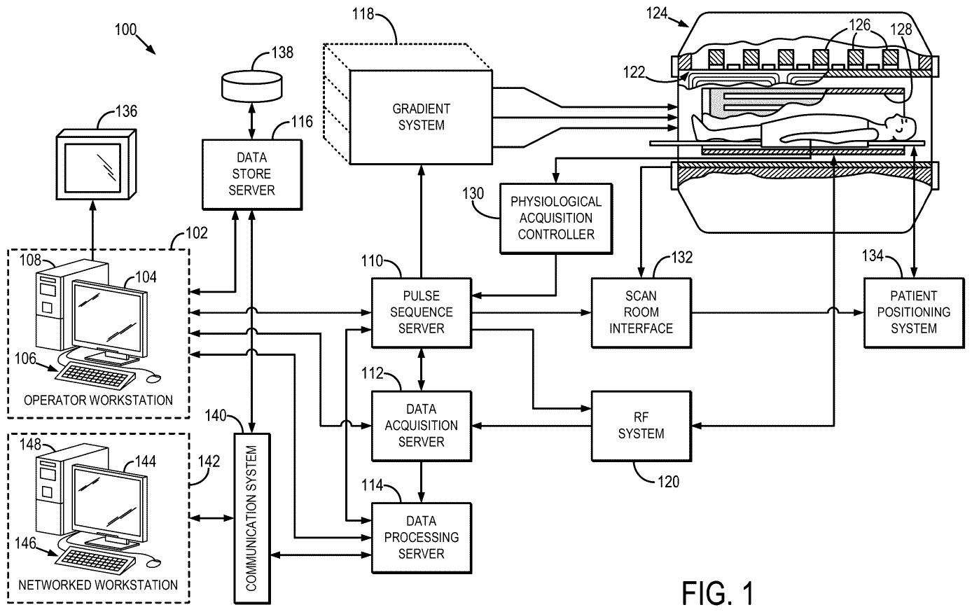

Referring particularly now to FIG. 1, an example of an MRI system 100 that can implement the methods described here is illustrated. The MRI system 100 includes an operator workstation 102 that may include a display 104, one or more input devices 106 (e.g., a keyboard, a mouse), and a processor 108. The processor 108 may include a commercially available programmable machine running a commercially available operating system. The operator workstation 102 provides an operator interface that facilitates entering scan parameters into the MRI system 100. The operator workstation 102 may be coupled to different servers, including, for example, a pulse sequence server 110, a data acquisition server 112, a data processing server 114, and a data store server 116. The operator workstation 102 and the servers 110, 112, 114, and 116 may be connected via a communication system 140, which may include wired or wireless network connections.

The pulse sequence server 110 functions in response to instructions provided by the operator workstation 102 to operate a gradient system 118 and a radiofrequency (“RF”) system 120. Gradient waveforms for performing a prescribed scan are produced and applied to the gradient system 118, which then excites gradient coils in an assembly 122 to produce the magnetic field gradients Gₓ, G_{y}, and G_{z} that are used for spatially encoding magnetic resonance signals. The gradient coil assembly 122 forms part of a magnet assembly 124 that includes a polarizing magnet 126 and a whole-body RF coil 128.

RF waveforms are applied by the RF system 120 to the RF coil 128, or a separate local coil to perform the prescribed magnetic resonance pulse sequence. Responsive magnetic resonance signals detected by the RF coil 128, or a separate local coil, are received by the RF system 120. The responsive magnetic resonance signals may be amplified, demodulated, filtered, and digitized under direction of commands produced by the pulse sequence server 110. The RF system 120 includes an RF transmitter for producing a wide variety of RF pulses used in MRI pulse sequences. The RF transmitter is responsive to the prescribed scan and direction from the pulse sequence server 110 to produce RF pulses of the desired frequency, phase, and pulse amplitude waveform. The generated RF pulses may be applied to the whole-body RF coil 128 or to one or more local coils or coil arrays.

The RF system 120 also includes one or more RF receiver channels. An RF receiver channel includes an RF preamplifier that amplifies the magnetic resonance signal received by the coil 128 to which it is connected, and a detector that detects and digitizes the I and Q quadrature components of the received magnetic resonance signal. The magnitude of the received magnetic resonance signal may, therefore, be determined at a sampled point by the square root of the sum of the squares of the I and Q components:

M=\sqrt{I²+Q²} (1);
and the phase of the received magnetic resonance signal may also be determined according to the following relationship:

$$φ = {tan}^{- 1} ( \frac{Q}{I} ) . \qquad ( 2 )$$

The pulse sequence server 110 may receive patient data from a physiological acquisition controller 130. By way of example, the physiological acquisition controller 130 may receive signals from a number of different sensors connected to the patient, including electrocardiograph (“ECG”) signals from electrodes, or respiratory signals from a respiratory bellows or other respiratory monitoring devices. These signals may be used by the pulse sequence server 110 to synchronize, or “gate,” the performance of the scan with the subject's heartbeat or respiration.

The pulse sequence server 110 may also connect to a scan room interface circuit 132 that receives signals from various sensors associated with the condition of the patient and the magnet system. Through the scan room interface circuit 132, a patient positioning system 134 can receive commands to move the patient to desired positions during the scan.

The digitized magnetic resonance signal samples produced by the RF system 120 are received by the data acquisition server 112. The data acquisition server 112 operates in response to instructions downloaded from the operator workstation 102 to receive the real-time magnetic resonance data and provide buffer storage, so that data is not lost by data overrun. In some scans, the data acquisition server 112 passes the acquired magnetic resonance data to the data processor server 114. In scans that require information derived from acquired magnetic resonance data to control the further performance of the scan, the data acquisition server 112 may be programmed to produce such information and convey it to the pulse sequence server 110. For example, during pre-scans, magnetic resonance data may be acquired and used to calibrate the pulse sequence performed by the pulse sequence server 110. As another example, navigator signals may be acquired and used to adjust the operating parameters of the RF system 120 or the gradient system 118, or to control the view order in which k-space is sampled. In still another example, the data acquisition server 112 may also process magnetic resonance signals used to detect the arrival of a contrast agent in a magnetic resonance angiography (“MRA”) scan. For example, the data acquisition server 112 may acquire magnetic resonance data and processes it in real-time to produce information that is used to control the scan.

The data processing server 114 receives magnetic resonance data from the data acquisition server 112 and processes the magnetic resonance data in accordance with instructions provided by the operator workstation 102. Such processing may include, for example, reconstructing two-dimensional or three-dimensional images by performing a Fourier transformation of raw k-space data, performing other image reconstruction algorithms (e.g., iterative or backprojection reconstruction algorithms), applying filters to raw k-space data or to reconstructed images, generating functional magnetic resonance images, or calculating motion or flow images.

Images reconstructed by the data processing server 114 are conveyed back to the operator workstation 102 for storage. Real-time images may be stored in a data base memory cache, from which they may be output to operator display 102 or a display 136. Batch mode images or selected real time images may be stored in a host database on disc storage 138. When such images have been reconstructed and transferred to storage, the data processing server 114 may notify the data store server 116 on the operator workstation 102. The operator workstation 102 may be used by an operator to archive the images, produce films, or send the images via a network to other facilities.

The MRI system 100 may also include one or more networked workstations 142. For example, a networked workstation 142 may include a display 144, one or more input devices 146 (e.g., a keyboard, a mouse), and a processor 148. The networked workstation 142 may be located within the same facility as the operator workstation 102, or in a different facility, such as a different healthcare institution or clinic.

The networked workstation 142 may gain remote access to the data processing server 114 or data store server 116 via the communication system 140. Accordingly, multiple networked workstations 142 may have access to the data processing server 114 and the data store server 116. In this manner, magnetic resonance data, reconstructed images, or other data may be exchanged between the data processing server 114 or the data store server 116 and the networked workstations 142, such that the data or images may be remotely processed by a networked workstation 142.

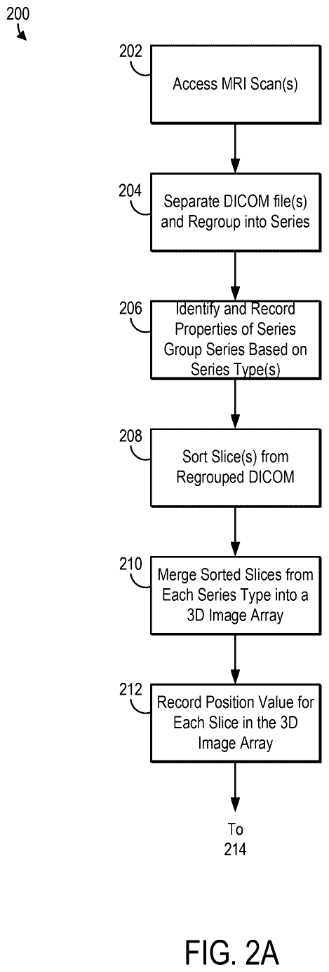

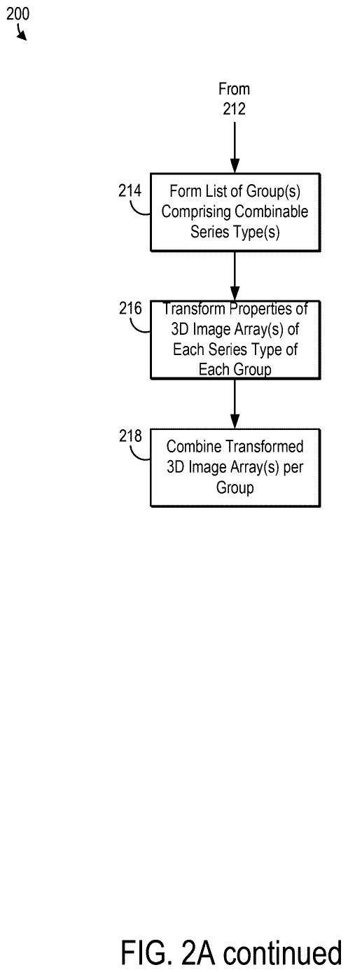

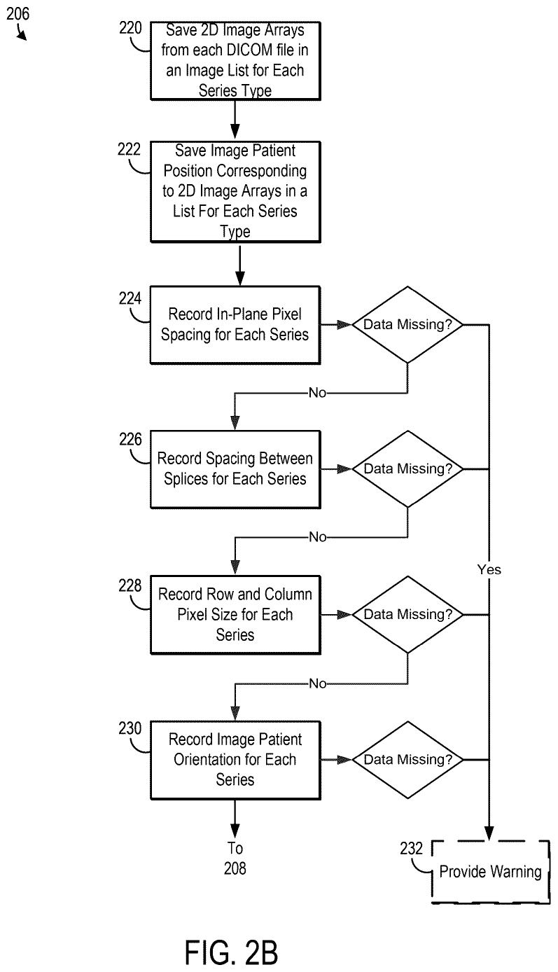

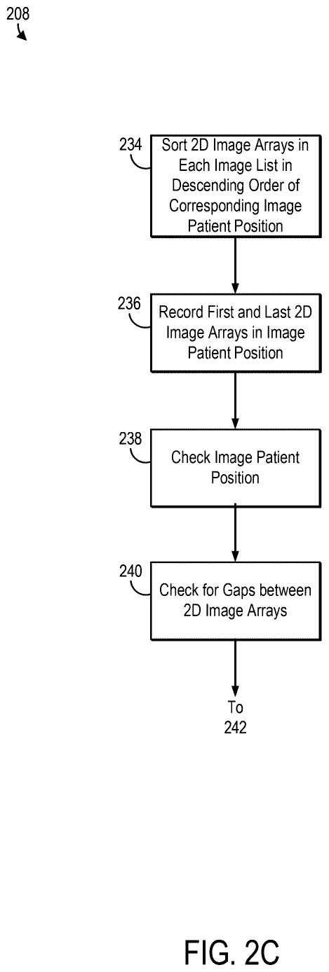

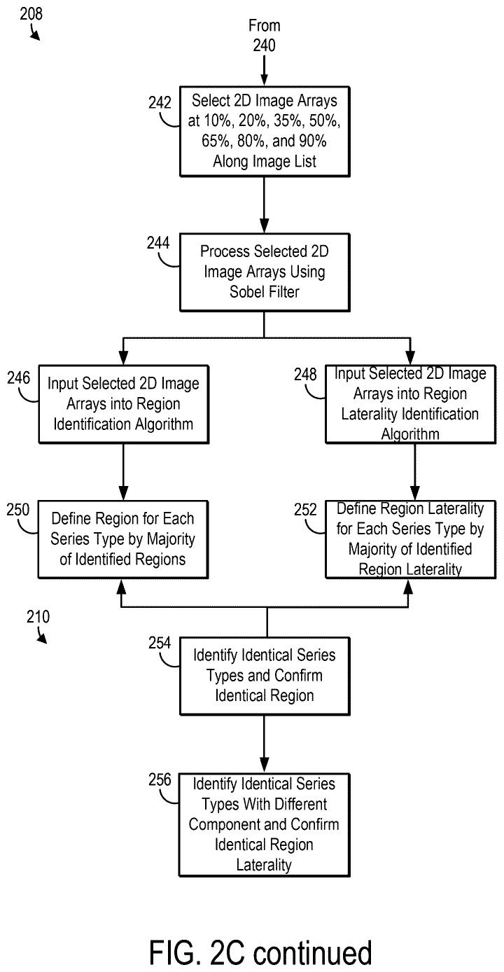

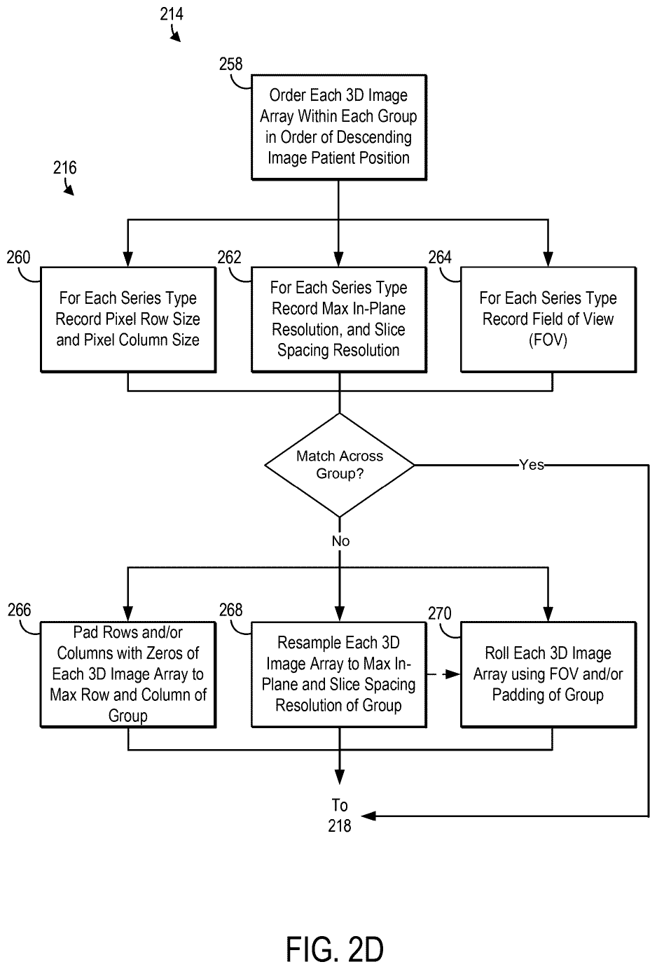

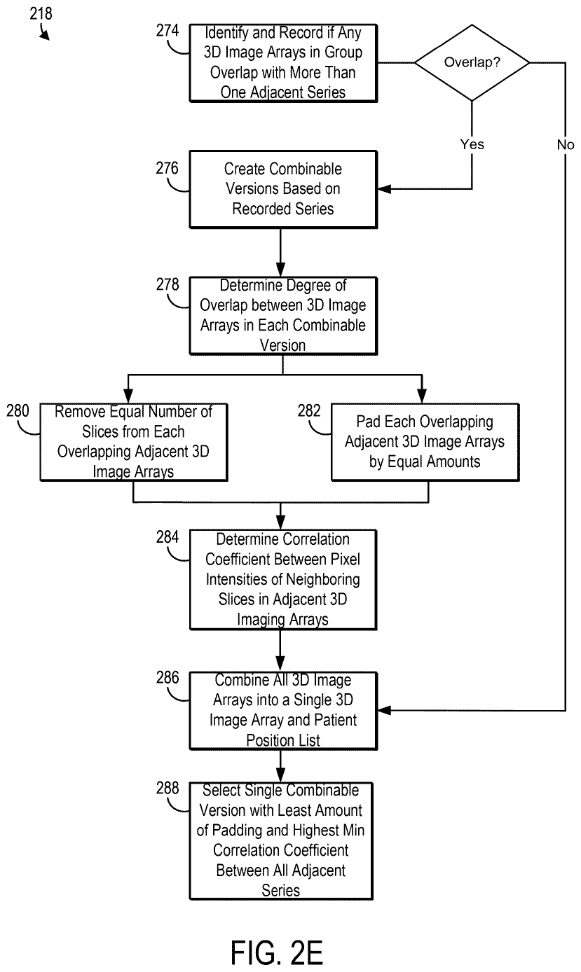

Referring now to FIG. 2A, an overview flowchart of non-limiting example steps 200 is shown for a preprocessing of MRI scans of a subject. The process 200 may be fully automated to conveniently provide an extended and detailed view of the musculoskeletal system to users. The process 200 can be completed on a suitable computer system.

First, one or more MRI scans are accessed by a processor at step 202. The MRI scans include DICOM files containing DICOM metadata. In a non-limiting example, each MRI scan includes DICOM metadata from one single study instance unique identifier (UID). Each DICOM file further includes a two-dimensional (2D) image array slice. As used herein, “slice” and “2D image array” may be used interchangeably.

At step 204, the DICOM files accessed by the processor are separated and regrouped into series. DICOM meta data further includes series instance UIDs, which may be vendor-specific. For example, in Philips scanners, all DICOM files of different component or phase designations may be stored as one series UID. In this example, step 204 would separate the Philips-acquired DICOM files into separate water, fat, in-phase, and out-of-phase series, and regroups each DICOM file into the separate component designations.

Next, the processor identifies and records one or more properties related to the one or more series and groups the series based on or more properties into different series types at step 206. In a non-limiting example, the series properties and series types include, but are not limited to, a weighting (e.g., T1, T2, unknown), fat saturation (e.g., fat saturation, no fat saturation, not applicable, or unknown), component (water, fat, in-phase, out-of-phase, not applicable, or unknown), resolution, derived/composed setting (e.g., non-derived/non-composed, derived/non-composed, composed/non-derived, composed/derived, or unknown), image patient position or acquisition plane (e.g., axial, sagittal, coronal, or unknown), patient orientation (e.g. head first supine (HFS), feet first supine (FFS), or unknown), subject region (e.g., lower body as anything below the upper pelvis, or upper body as anything above the upper pelvis), subject region laterality (e.g., bilateral, left, right, or unknown), or any combination thereof. The processor is capable of identifying these properties either every image in the MRI series is saved as its own DICOM file, or if the series is saved as a single DICOM file and the meta data houses the nested image by image information (“multi-frame stack”).

In a non-limiting example, the processor is configure to identify and exclude DICOM files from (1) a non-MRI modality, (2) DICOM files that are a survey, localizer, scout series, calibration, or summary series, (3) DICOM files that are not an image series, (4) DICOM files where the image series is not 3D, or (4) any combination thereof. For example, in (2), the processor may identify keywords in series descriptions, series containing only three images, or if images in a series are from different image patient positions.

At step 208, the slices are sorted from the regrouped DICOM files by series type in order of each slice's location along an image patient position of the patient. The image patient position represents the 2D cut plane for the patient including axial, sagittal, coronal or oblique planes. Each slice is a 2D image array, where pixels arranged horizontally are defined as rows and pixels arranged vertically are defined as columns with respect to the plane in which the images were collected. Thereafter at step 210, the sorted slices in each series type are merged into a single three-dimensional (3D) image array, and a position value for each slice in the 3D image array is recorded at step 212.

Next, the processor forms a list of one or more groups comprising the 3D image arrays that are combinable based on sharing the same series type and a location of the patient at step 214. As used herein, the “location of the patient” refers to situations where the origin for the patient orientation relative to the patient did not change.

At step 216, the processor transforms one or more features of the one or more 3D image arrays of each series type to be identical. In a non-limiting example, the features include, but are not limited to, pixel row and column size, FOV, maximum in-plane resolution, or identical slice spacing resolution.

The transformed 3D image arrays are combined at step 218 using the position values of each slice of the 3D image arrays and a slice spacing. As will be described in further detail below, slices from adjacent 3D image arrays may need to be removed or added to produce a single, continuous 3D image array. The processor may further record the combined 3D image array's final attributes including, but not limited to, 3D pixel resolution and an image patient position of the first image slice.

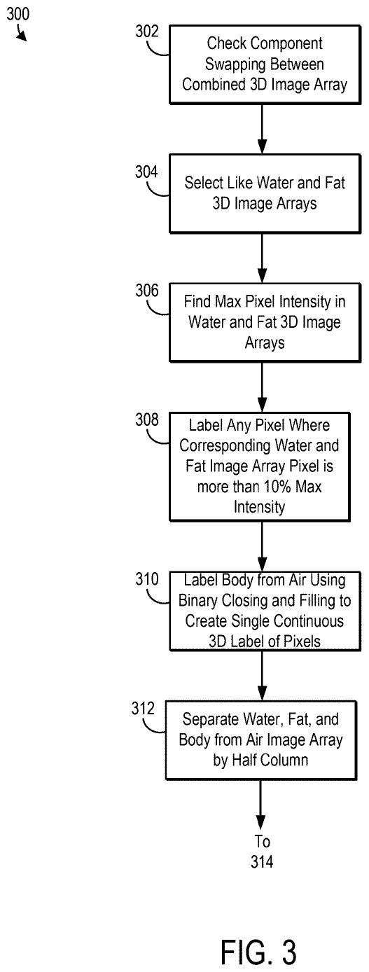

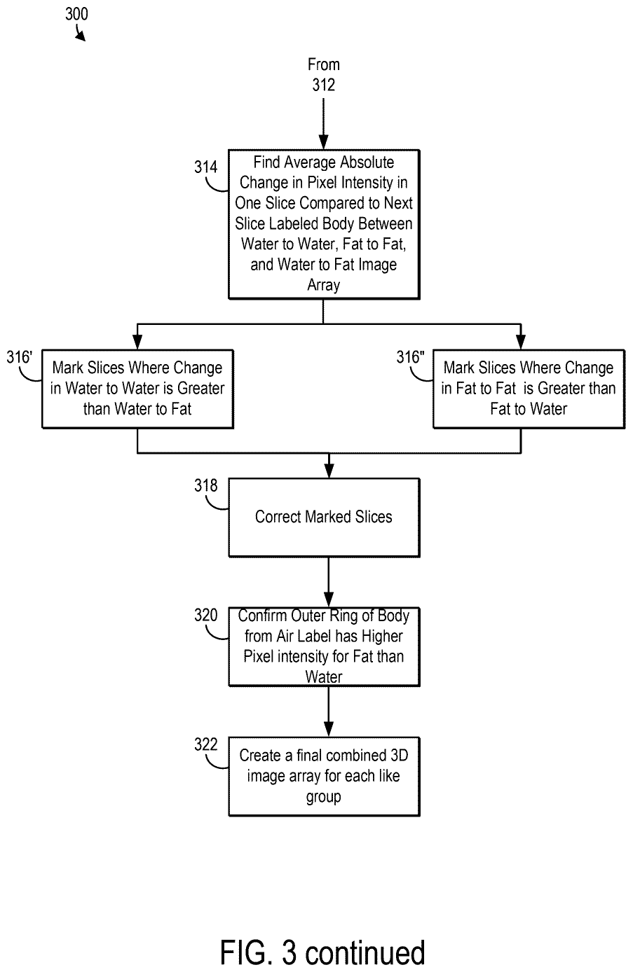

As will be described in FIG. 3 below, the processor may further check and correct any swapping between a fat component and water component between two combined 3D images of the same series type.

Referring now to FIG. 2B, detailed steps of broadly defined step 206 are provided. Specifically, identifying and recording properties related to the series and series types includes first saving the 2D image arrays from each DICOM file in an image list for each series type at step 220. Thereafter or substantially concurrently, the processor further saves an image patient position corresponding to the 2D image arrays in an image patient position list for each of the series types at step 222. Each DICOM file provides the image patient position in the axial, sagittal, and coronal planes for the top left most pixel in each 2D image array. In a non-limiting example, each 2D image array and corresponding image patient position are saved in their respective lists in the same order.

Next, at steps 224-230, the in-plane pixel spacing, spacing between splices, row and column pixel size, and image patient position are recorded by the processor for each 2D image array of each series type, respectively. Step 224-230 may occur sequentially or substantially concurrently. Additionally, steps 224-230 may occur in any order and is not limited to the order shown in FIG. 2B. If any of the information in steps 224-230 is missing, the processor may optionally provide a warning to a user indicating the missing information in step 232. In a non-limiting example, the slice thickness may be used in instances where the spacing between the splices is not given or is 0 at step 226.

Referring now to FIG. 2C, detailed steps of broadly defined steps 208 and 210 are provided. In a non-limiting example, the workflow in FIG. 2C continues directly after step 230. At step 234, the 2D image arrays in each image list are sorted. In a non-limiting example, the 2D image arrays are sorted in descending order of their corresponding image patient position. In a non-limiting example, each image list includes a series direction defined by the axial, sagittal, or coronal acquisition planes. Therefore, the 2D image arrays are organized in each image list according to the image slice position. Next, at step 236, the processor records the first 2D image array and the last 2D image array in each image list, corresponding to a series type. As used herein, each image list marked with the first 2D image array and last 2D image array defines a series slice coverage or a series list coverage.

At step 238, the processor may further check the image patient position to determine if the images need to be flipped in any of the acquisition planes to maintain the image presentation of the patient as follows: anterior to posterior down the rows, right to left patient side along the columns, and inferior to superior with increasing slices. Next at step 240, the processor may further check that there is no ‘gaps’ between images that would indicate a missing file, using the spacing between slices and the sorted image patient position for the slice plane. For example, if there is a slice spacing of 5 mm and patient position goes from 0 mm to 5 mm, 5 mm to 10 mm, 10 mm to 20 mm, and 20 mm to 25 mm, there is an indication of a missing slice between 10 mm and 20 mm. Any missing slices will be logged and a blank image (with 0 intensity) will be used as a placeholder so the array of slices remains continuous.

After the 2D image arrays have been sorted for each list, the processor further defines the region and region laterality of the series type of each image list in steps 242-256. In a non-limiting example, the region and region laterality may include those defined previously.

At step 242, the processor may select a plurality of 2D image arrays in each series type at one or more predefined intervals along the image list. For example, 2D image arrays from each list may be selected at, but not limited to, 1%, 2%, 5%, 10%, or 15% intervals. In a non-limiting example, 2D image arrays from may be selected at 10%, 20%, 35%, 50%, 65%, 80%, and 90% along the image list.

In a non-limiting example, each selected 2D image array from step 242 may be further processed using a Sobel filter at step 244. In this step, the data from the 2D image array is transformed to provide an outline of the imaged region and minimizing the effects of image type and contrast variation. The filtered, selected 2D image arrays are then input into a region identification algorithm using AI or image processing to label each selected slice as an identified patient region (step 246) and a region laterality identification algorithm using AI or image processing to label each selected slice as an identified patient region laterality (step 248). In a non-limiting example, the region identification algorithm using AI is trained using slices of the lower body region and the upper body region. In a non-limiting example, the region laterality identification algorithm using AI is trained using slices of bilateral, left, or right coverage.

Next, at step 250, the processor defines the series type of each image list based on the designation of the majority of output slices from the region identification algorithm. Likewise at step 252, the processor defines the series type of each image list based on the designation of the majority of output slices from the region laterality identification algorithm.

Thereafter, the processor merges the sorted 2D image arrays in each defined image list into a 3D image array, and, at step 254, may further identify image lists of identical series types except for different components and confirm they share identical region designations. For example, the processor may identify two image lists that both have T1 weightings, no fat saturation, are non-composed and non-derived, have HFS patient orientation, and show 2D image arrays in the axial plane but one has a water component and the other has a fat component. The processor would confirm whether these to image lists differing in component share the same region. In the event the processor determines that the do not share the same region, the processor may select 2D image arrays at an interval along the image lists for all like series types and repeat steps 246-252 until the region matches for all like series types. In a non-limiting example, the interval may be, but is not limited to, 1%, 2%, 5%, 10%, or 15% intervals. For example, the interval may be 10%. The processor may further record any discrepancies between like series types.

Likewise at step 256, the processor may identify image lists of identical series types except for different components and confirm they share identical region laterality designations. In the event the processor determines that they do not share the same region laterality, the processor may take each of the identified 3D image arrays and label the patient's body from air in each 2D slice. For example, each 2D image array's pixel intensities are divided by its maximum intensity in the array, where all pixels above 0.1 are labeled “body”. Next, the labeled pixels in each of the identified lists are then post-processed to create a single solid label using binary closing, binary filling, and constraining it to being one single continuous 3D label across the array of 2D images. The processor may then take the column-wise center of mass of the 3D label for each like series type and find the average column center of mass. If it is more towards the right side of the array column-wise, label it “right” and if more to the left, label all like series region laterality as “left”.

In a non-limiting example, the series splice coverage or list splice coverage from step 236 is carried over into the merged 3D image array as captured by step 212.

Referring now to FIG. 2D, detailed steps of broadly defined step 214 are provided. Specifically, after the processor has formed a list of groups of combinable 3D image arrays based on shared series type, the processor is configured to order each 3D image array within each group by descending image patient position at step 258. To be combinable, the 3D image arrays within each group must share a weighting, fat saturation, component, derived/composed setting, image patient position, patient orientation, region, and region laterality. Note, these steps are one non-limiting example method to quantify the region and region laterality of each group. This is done to ensure that groups, when combined, share the same image reference frame (i.e. the position assigned for the image location is in the same relative position to the patient's anatomy) from which MRI data was collected while also adding detail as to what that reference frame captured is (for an example, the arm or legs, the left side or both sides are captured). Alternatively, the DICOM tag “Frame of Reference UID” can be used instead as the region and region laterality value, in which groups of images/series sharing the same value indicate they capture the same region and may be able to be combined. However, this method depends on the technician/software to properly update the MRI acquisition as a series being located in a new reference frame whenever it changes (patient moves or new region is being captured with a new reference regarding where the images are being captured in relation to the patient), and does not provide any information regarding what region of the patient is within the coverage.

Before combining the grouped 3D image arrays, one or more transformations of the 3D image array data may be required as provided broadly in step 216. Specifically, at steps 260-264, the processor records the following features of each 3D image array in a group: the pixel row size, the pixel column size, the maximum in-plane resolution, the slice spacing resolution, and the FOV for each 3D image. In a non-limiting example, the FOV is the average position of the patient in the row and column acquisition plane. The processor may then confirm whether the recorded features in steps 260-264 match across the group. If features match for all combinable 3D image arrays in a group, the processor continues to combine the groups per step 218. However, if any feature of the 3D image arrays does not match with group, one of the following three steps occur, or any combination of the three steps: At step 266, the processor pads the rows and/or columns of one or more 3D image arrays in the group to match the maximum row and column of the group. At step 268, the processor may resample each 3D image array in the group to match the maximum in-plane resolution and slice-spacing resolution of the group. At step 270, the processor may roll each 3D image array using the FOV and/or padding of step 268 to match the average FOV across all 3D image arrays of the group.

Referring now to FIG. 2E, detailed steps of the broadly defined combining step 218 are provided. Specifically, the processor may identify and record in any 3D image arrays with a group overlap with any other within the series splice coverage beyond just the first slice and the last slice at step 274. If there is no overlap beyond the first slice and last slice between adjacent 3D image arrays in a group, then the process may skip to step 286 which will be described in further detail below. However, if there is an overlap in the series slice coverage, the processor may record the overlapping 3D image arrays and create combinable versions based on the recorded, overlapping 3D image arrays at step 276. This could indicate a repeated series or a series that was taken again after patient movement. In a non-limiting example, this may indicate that one or more of these 3D image arrays should be removed to make the most continuous and accurate combination of all the 3D image arrays. In a non-limiting example, the combinable versions include combinable versions include a version in which all recorded, overlapping 3D image arrays in the group are used, one or more versions in which all but one of the recorded, overlapping 3D image arrays are used, and a version in which none of the recorded, overlapping 3D image arrays are used. For each version of combinable series, the spacing between slices and the series slice coverage are used to combine the adjacent 3D image arrays.

Next, at step 278, the processor determines a degree of overlap between adjacent 3D image arrays in each combinable version. If adjacent series overlap by more than one slice, remove equal amounts of slices from each series' 3D image array on their respective adjacent ends at step 280. On the other hand, if padding is needed, adjacent 3D image arrays may be padded by equal amounts at step 282.

At step 284, the processor further determines a correlation coefficient between pixel intensities of neighboring slices in adjacent 3D imaging arrays. In a non-limiting example, only the pixels labeled as the patient body are used in both 3D imaging arrays using the method of labeling body from air described but applied to a single image. Thereafter, all combinable versions are combined into a single 3D image array at step 286.

If multiple combinable versions were created, the processor is configured to a single combinable version with the least amount of padding added and the highest minimum correlation coefficient between all adjacent 3D image arrays in each group. For the final version of the combined 3D image arrays, the processor may record the final maximum row size, column size, spacing between slices, and in-plane resolution as the now combined 3D image array attributes. Use the final combined 3D image array and image patient position respectively. Save the shared attributes for all series in the “series to combine” such as weighting, fat saturation, component, etc. The minimum and maximum image patient position may be used in the slice plane as the combined 3D image array slice coverage.

The processor may further combine lower body and upper body combined 3D image arrays by repeating the workflow starting at step 276.

Referring now to FIG. 3, a workflow 300 for checking and correcting component swapping is described. At step 302, the processor checks component swapping between the combined 3D image arrays of step 288. The processor may select like water and fat combined 3D image arrays at step 304. At step 306, the processor may find the maximum pixel intensity in each of the like water and fat combined 3D image arrays. At step 308, the processor may label any pixel where the corresponding water and fat image array pixel is more than 10% of the maximum intensity found in the previous step. At step 310, binary closing and filling may be used to create a single continuous 3D label of pixels to label the body of the patient from air.

At step 312, water, fat, and body are separated from air in the 3D image arrays half column-wise, where each half is treated as the left and right side respectively. For each half, steps 314-320 are performed. At 314, the processor evaluates the water, fat, and body from air in each 3D image array slice by slice and finds the average absolute change in pixel intensities at each slice compared to the next slice labeled body between the water to water, fat to fat, and water to fat image array. At step 316′, the processor marks any slices where the change in water to water is greater than the change in water to fat as a transition slice. Likewise, at step 316″, the processor marks any slice where the change in fat to fat is greater than the change in fat to water as a transition slice. The transition slices indicate that a component swap occurred. Thereafter, at step 318, the transition slices are corrected. Depending on if there is an odd or even amount of transitions, the alternating transitions can be either water to fat or fat to water. In a non-limiting example, it can be tested by assuming one version of the transition (swap fat with the water image array from these transition brackets and water with fat).

At step 320, the processor may further confirm after the switch that the outer ring of the body from air label has a higher pixel intensity for fat than water (as it should for subcutaneous fat), otherwise, use other transition.

At step 322, a final combined 3D image array may be created for each like group. In a non-limiting example, the final combined 3D image array may be saved as a Neuroimaging Informatics Technology Initiative (NIfTI) file. Further, the final combined 3D image array may be used as the pixel data, the image patient position may be set to default (as collection position will be saved in the name to indicate the image patient position), the final in-plane resolution and slice spacing resolution may be used as the image resolution, and the first slice image patient position may be used as the origin. In a non-limiting example, each group may be named to include the following designations:

Weighting: Not applicable (NA), T1, T2, or unknown (UK)

Fat Saturation: NA, fat saturation (FS), no FS, or UK

Component or phase: NA, water (W), fat (F), in-phase (IP), out-of-phase (OP), or UK

Derived/Composed Settings: non-derived (ND), derived (D), composed/non-derived (CND), composed/derived (CD), or UK

Image Patient Position or Plane of Acquisition: axial (AX), sagittal (SA), coronal (CO), or UK

Patient Orientation: head first supine (HFS), feet first supine (FFS), or UK

Region: lower extremity (LE) covering both lengths, upper body (UB), and extended lower extremity (LE_EXT) covering both legs and the paraspinal muscles in the lower abdomen region.

Region laterality: bilateral (BILAT), left (L), right (R), or UK

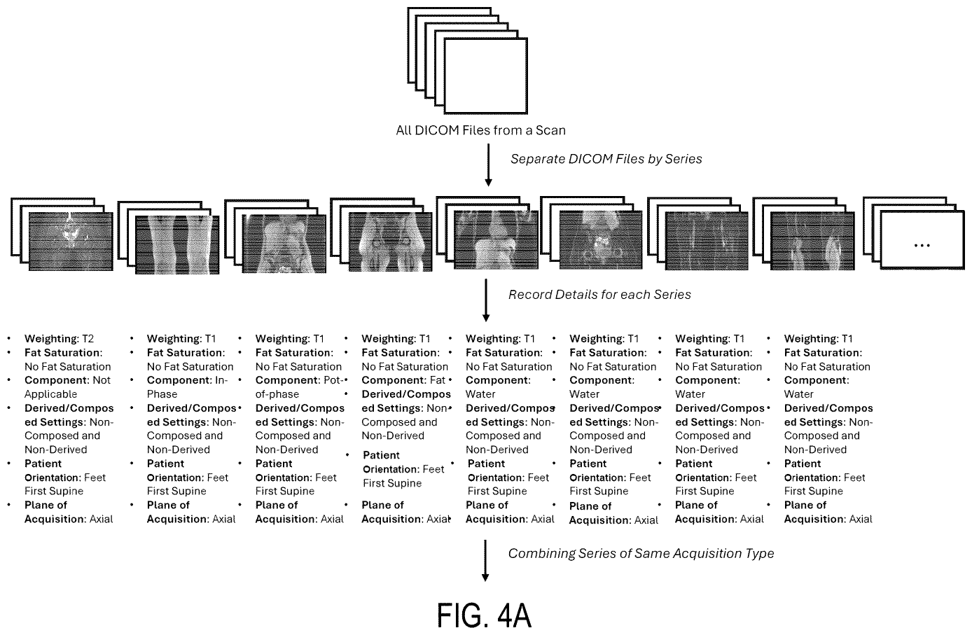

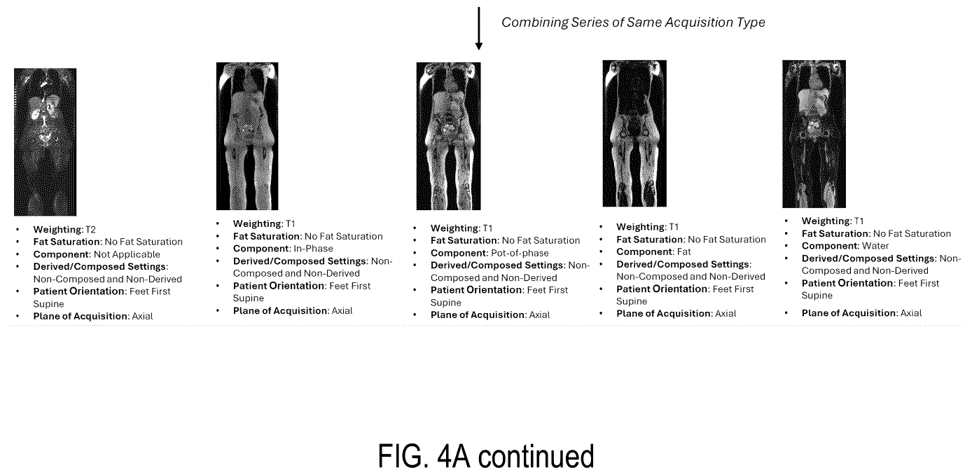

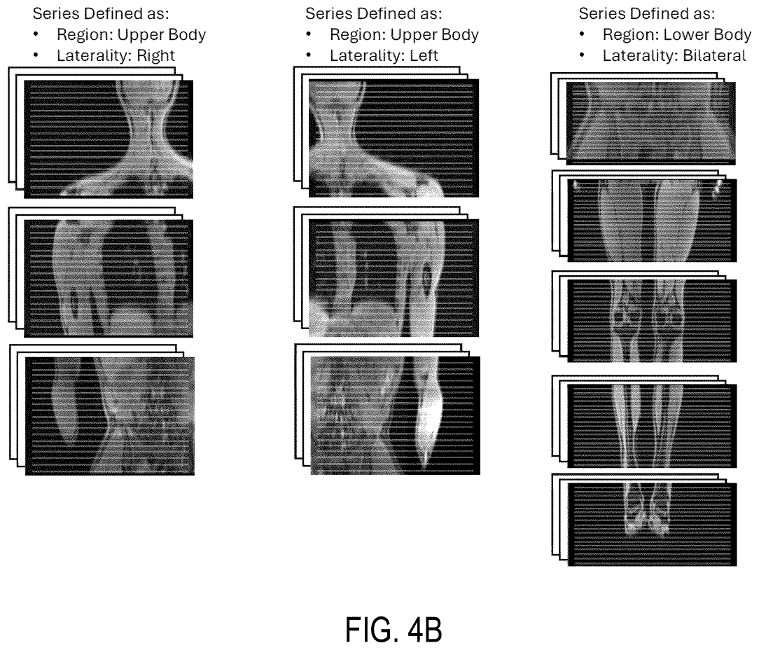

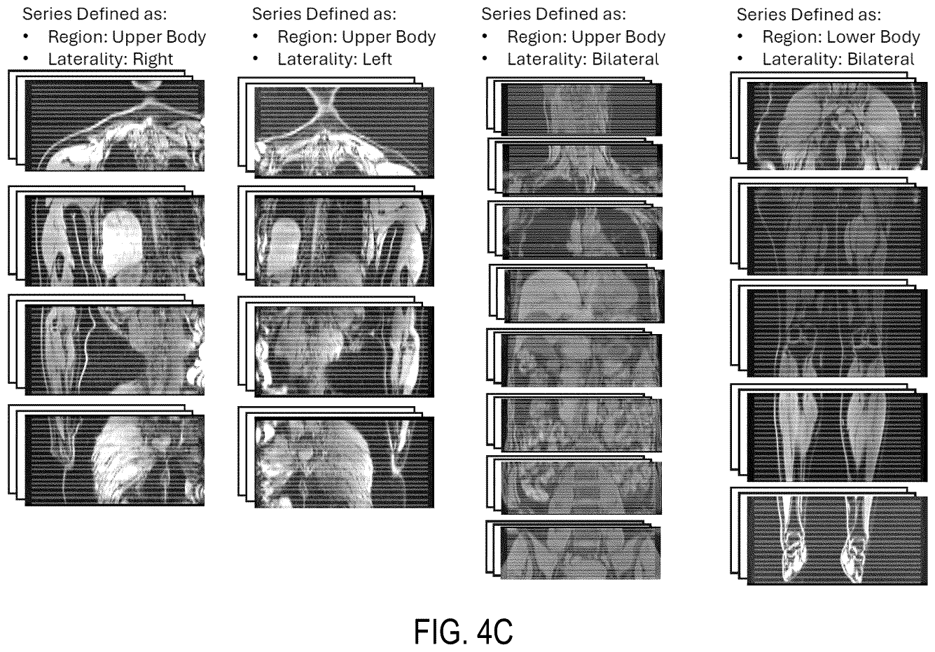

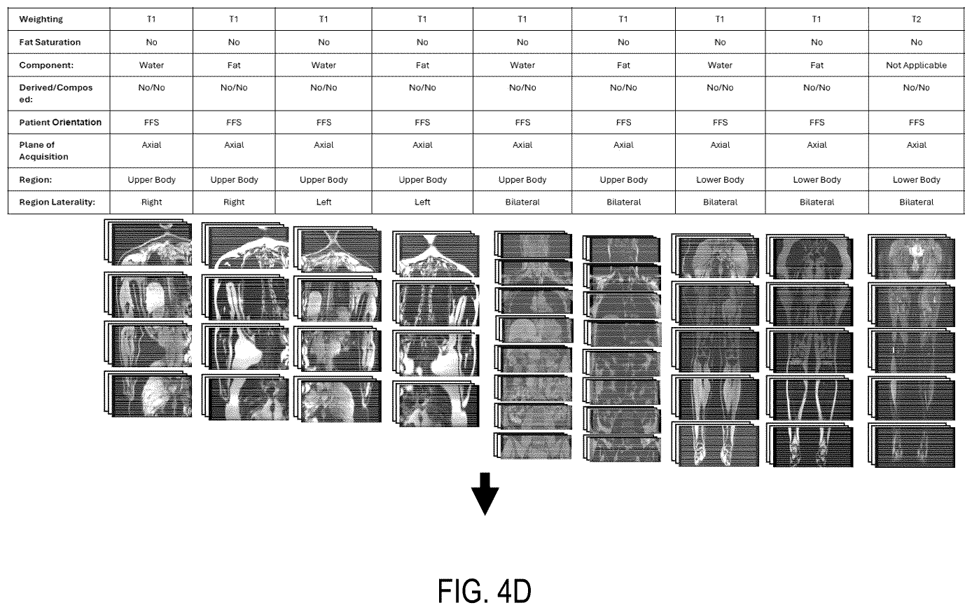

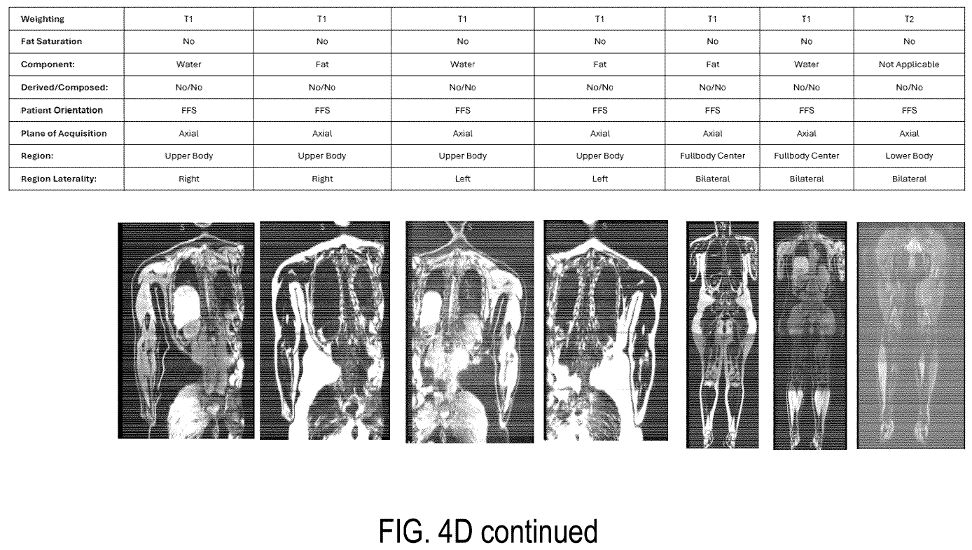

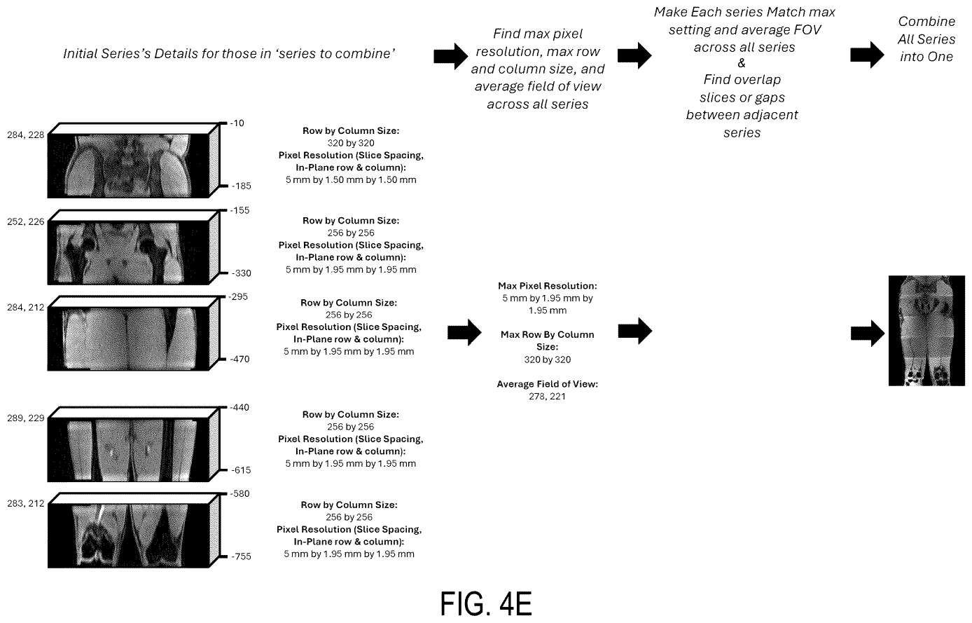

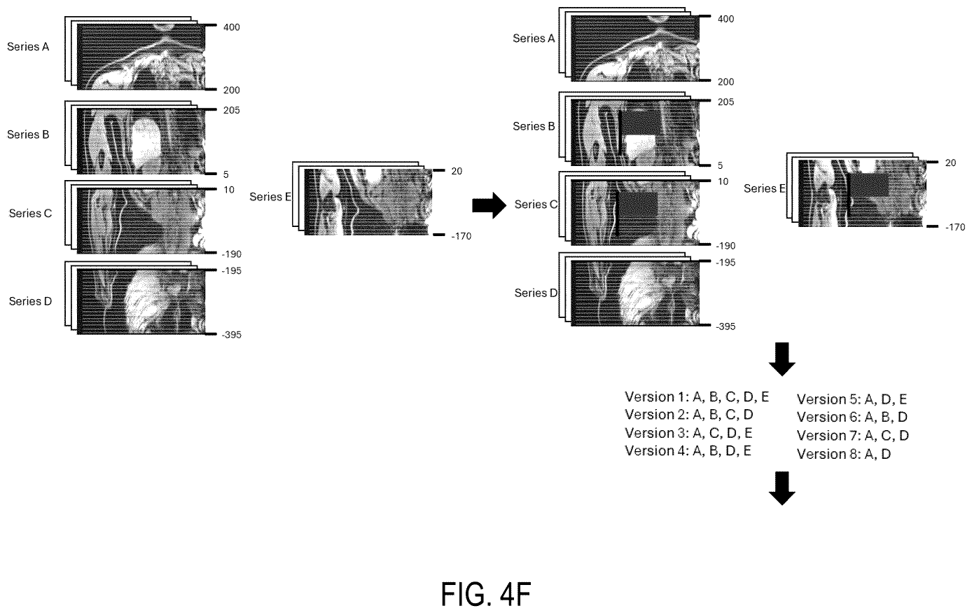

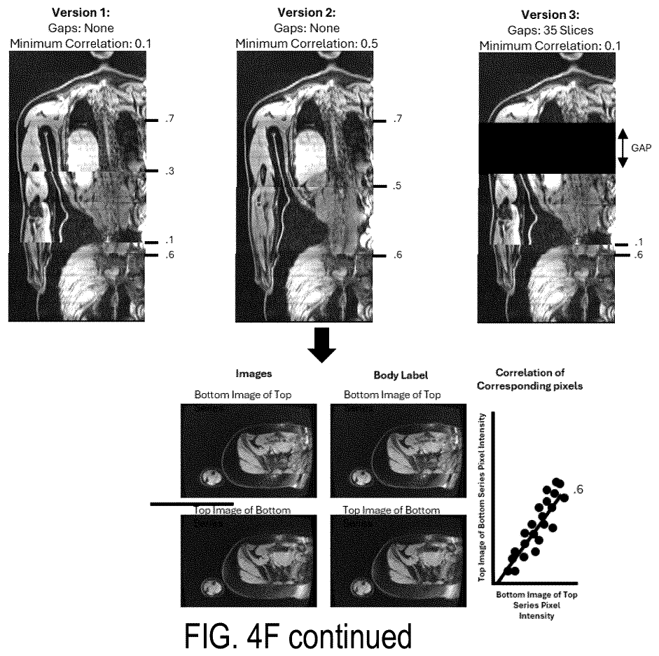

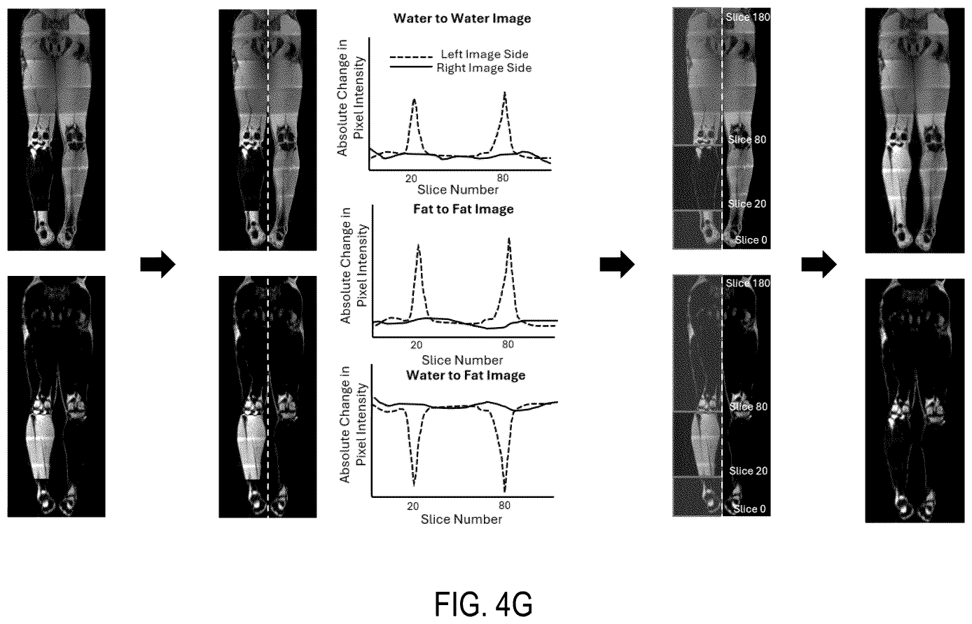

Non-limiting examples of the workflows described above are shown in FIGS. 4A-4F.

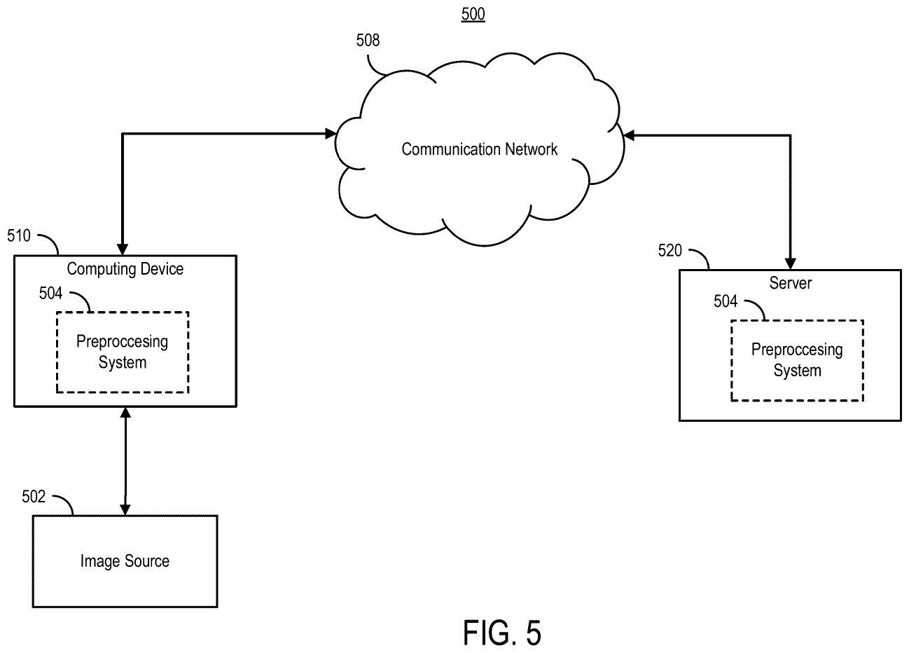

The above-described systems and methods may be implemented using a variety of systems and networks. FIG. 5 shows an example 500 of a system for automatically carrying out the processes described above. As shown in FIG. 5, a computing device 510 can receive multiple types of image data from an image source 502. In some configurations, computing device 510 can execute at least a portion of a preprocessing system 504 as described above.

Additionally or alternatively, in some embodiments, computing device 510 can communicate information about image data received from image source 502 to a server 520 over a communication network 508, which can execute at least a portion of the preprocessing system 504. In such embodiments, server 520 can return information to computing device 510 (and/or any other suitable computing device) indicative of an output of the preprocessing system 504.

In some embodiments, computing device 510 and/or server 520 can be any suitable computing device or combination of devices, such as a desktop computer, a laptop computer, a smartphone, a tablet computer, a wearable computer, a server computer, a virtual machine being executed by a physical computing device, etc. In some configurations, the preprocessing system 504 can extract features from labeled (e.g., labeled as including a condition or disease, or normal) image data using a convolutional neural network (CNN), UNet, and the like, trained as a general image classifier, and can perform a correlation analysis to calculate correlations between the features corresponding to the image data and a database. In some embodiments, the labeled data can be used to train a classification model, such as a support vector machine (SVM), to classify features as indicative of bone, tissue, or fat. In some embodiments, automatic segmentation system 504 can provide features for unlabeled image data to the trained classification model and can present a quantitative assessment, such as a fat infiltration score or ratio based on the output of the classification model (e.g., based on which class the SVM identifies the features with).

In some embodiments, image source 502 can be any suitable source of image data, such as an MRI, CT, or another computing device (e.g., a server storing image data), etc. In a preferred embodiment, the image source is an MRI system. In some embodiments, image source 502 can be local to computing device 510. For example, image source 502 can be incorporated with computing device 510 (e.g., computing device 510 can be configured as part of a device for capturing and/or storing images). As another example, image source 502 can be connected to computing device 510 by a cable, a direct wireless link, etc. Additionally or alternatively, in some embodiments, image source 502 can be located locally and/or remotely from computing device 510, and can communicate image data to computing device 510 (and/or server 520) via a communication network (e.g., communication network 508).

In some embodiments, communication network 508 can be any suitable communication network or combination of communication networks. For example, communication network 508 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), a wired network, etc. In some embodiments, communication network 508 can be a local area network, a wide area network, a public network (e.g., the Internet), a private or semi-private network (e.g., a corporate or university intranet), any other suitable type of network, or any suitable combination of networks. Communications links shown in FIG. 5 can each be any suitable communications link or combination of communications links, such as wired links, fiber optic links, Wi-Fi links, Bluetooth links, cellular links, etc.

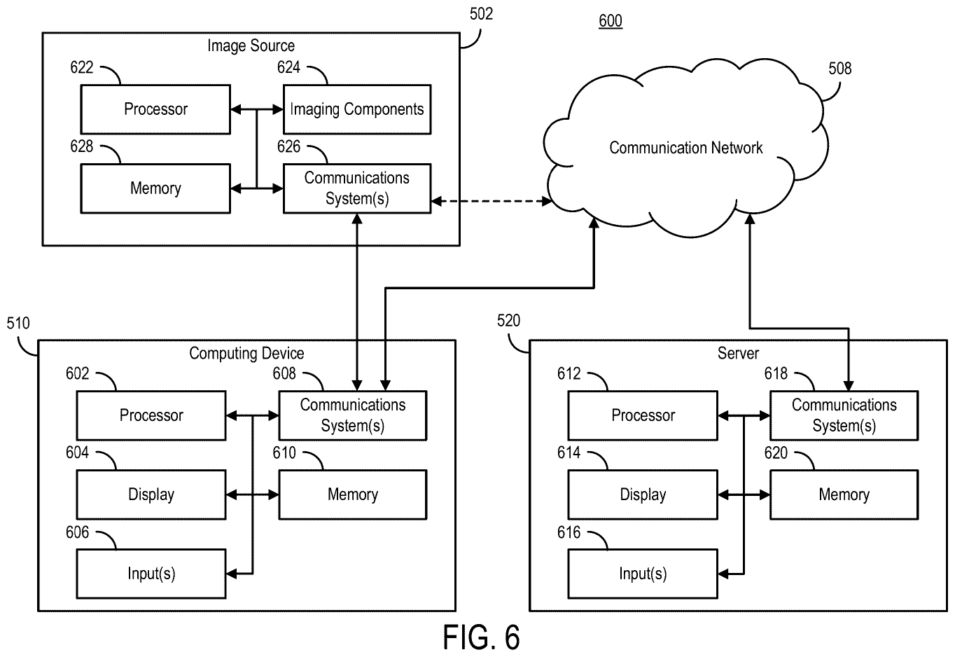

FIG. 6 shows an example 600 of hardware that can be used to implement image source 502, computing device 510, and/or server 520 in accordance with some embodiments of the disclosed subject matter. As shown in FIG. 6, in some embodiments, computing device 510 can include a processor 602, a display 604, one or more inputs 606, one or more communication systems 608, and/or memory 610. In some embodiments, processor 602 can be any suitable hardware processor or combination of processors, such as a central processing unit (CPU), a graphics processing unit (GPU), etc. In some embodiments, display 604 can include any suitable display devices, such as a computer monitor, a touchscreen, a television, etc. In some embodiments, inputs 606 can include any suitable input devices and/or sensors that can be used to receive user input, such as a keyboard, a mouse, a touchscreen, a microphone, etc.

In some embodiments, communications systems 608 can include any suitable hardware, firmware, and/or software for communicating information over communication network 508 and/or any other suitable communication networks. For example, communications systems 608 can include one or more transceivers, one or more communication chips and/or chip sets, etc. In a more particular example, communications systems 608 can include hardware, firmware and/or software that can be used to establish a Wi-Fi connection, a Bluetooth connection, a cellular connection, an Ethernet connection, etc.

In some embodiments, memory 610 can include any suitable storage device or devices that can be used to store instructions, values, etc., that can be used, for example, by processor 602 to present content using display 604, to communicate with server 520 via communications system(s) 608, etc. Memory 610 can include any suitable volatile memory, non-volatile memory, storage, or any suitable combination thereof. For example, memory 610 can include RAM, ROM, EEPROM, one or more flash drives, one or more hard disks, one or more solid state drives, one or more optical drives, etc. In some embodiments, memory 610 can have encoded thereon a computer program for controlling operation of computing device 510. In such embodiments, processor 602 can execute at least a portion of the computer program to present content (e.g., MRI images, user interfaces, graphics, tables, etc.), receive content from server 520, transmit information to server 520, etc.

In some embodiments, server 520 can include a processor 612, a display 614, one or more inputs 616, one or more communications systems 618, and/or memory 620. In some embodiments, processor 612 can be any suitable hardware processor or combination of processors, such as a CPU, a GPU, etc. In some embodiments, display 614 can include any suitable display devices, such as a computer monitor, a touchscreen, a television, etc. In some embodiments, inputs 616 can include any suitable input devices and/or sensors that can be used to receive user input, such as a keyboard, a mouse, a touchscreen, a microphone, etc.

In some embodiments, communications systems 618 can include any suitable hardware, firmware, and/or software for communicating information over communication network 508 and/or any other suitable communication networks. For example, communications systems 618 can include one or more transceivers, one or more communication chips and/or chip sets, etc. In a more particular example, communications systems 618 can include hardware, firmware and/or software that can be used to establish a Wi-Fi connection, a Bluetooth connection, a cellular connection, an Ethernet connection, etc.

In some embodiments, memory 620 can include any suitable storage device or devices that can be used to store instructions, values, etc., that can be used, for example, by processor 612 to present content using display 614, to communicate with one or more computing devices 510, etc. Memory 620 can include any suitable volatile memory, non-volatile memory, storage, or any suitable combination thereof. For example, memory 620 can include RAM, ROM, EEPROM, one or more flash drives, one or more hard disks, one or more solid state drives, one or more optical drives, etc. In some embodiments, memory 620 can have encoded thereon a server program for controlling operation of server 520. In such embodiments, processor 612 can execute at least a portion of the server program to transmit information and/or content (e.g., MRI data, results of automatic diagnosis, a user interface, etc.) to one or more computing devices 510, receive information and/or content from one or more computing devices 510, and/or receive instructions from one or more devices (e.g., a personal computer, a laptop computer, a tablet computer, a smartphone, etc.).

In some embodiments, image source 502 can include a processor 622, imaging components 624, one or more communications systems 626, and/or memory 628. In some embodiments, processor 622 can be any suitable hardware processor or combination of processors, such as a CPU, a GPU, etc. In some embodiments, imaging components 624 can be any suitable components to generate image data corresponding to one or more imaging modes (e.g., T1 imaging, T2 imaging, fMRI, etc.). An example of an imaging machine that can be used to implement image source 502 can include a conventional MRI scanner (e.g., a 1.5 T scanner, a 3 T scanner), a high field MRI scanner (e.g., a 7 T scanner), an open bore MRI scanner, a CT system, an ultrasound scanner and the like.

Note that, although not shown, image source 502 can include any suitable inputs and/or outputs. For example, image source 502 can include input devices and/or sensors that can be used to receive user input, such as a keyboard, a mouse, a touchscreen, a microphone, a trackpad, a trackball, hardware buttons, software buttons, etc. As another example, image source 502 can include any suitable display devices, such as a computer monitor, a touchscreen, a television, one or more speakers, etc.

In some embodiments, communications systems 626 can include any suitable hardware, firmware, and/or software for communicating information to computing device 510 (and, in some embodiments, over communication network 508 and/or any other suitable communication networks). For example, communications systems 626 can include one or more transceivers, one or more communication chips and/or chip sets, etc. In a more particular example, communications systems 626 can include hardware, firmware and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, etc.

In some embodiments, memory 628 can include any suitable storage device or devices that can be used to store instructions, values, image data, etc., that can be used, for example, by processor 622 to: control imaging components 624, and/or receive image data from imaging components 624; generate images; present content (e.g., MRI images, a user interface, etc.) using a display; communicate with one or more computing devices 510; etc.

Memory 628 can include any suitable volatile memory, non-volatile memory, storage, or any suitable combination thereof. For example, memory 628 can include RAM, ROM, EEPROM, one or more flash drives, one or more hard disks, one or more solid state drives, one or more optical drives, etc. In some embodiments, memory 628 can have encoded thereon a program for controlling operation of image source 502. In such embodiments, processor 622 can execute at least a portion of the program to generate images, transmit information and/or content (e.g., MRI image data) to one or more computing devices 510, receive information and/or content from one or more computing devices 510, receive instructions from one or more devices (e.g., a personal computer, a laptop computer, a tablet computer, a smartphone, etc.), and the like.

The present disclosure has described one or more preferred embodiments, and it should be appreciated that many equivalents, alternatives, variations, and modifications, aside from those expressly stated, are possible and within the scope of the invention.

1. A system for preprocessing magnetic resonance imaging (MRI) scans of a patient acquired with a medical imaging system, the system comprising:
a memory configured to store one or more MRI scans of a patient, wherein each MRI scan includes one or more Digital Imaging and Communications in Medicine (DICOM) files acquired from a single study instance unique identifier (UID), and wherein each DICOM file includes a two-dimensional (2D) image array slice;
a processor configured to:
(i) access the one or more MRI scans;
(ii) separate the one or more DICOM files grouped based on one or more series instance UIDs and regroup the one or more DICOM files into one or more series;
(iii) identify and record one or more properties related to the one or more series and group the series based on one or more proprieties into different series types;
(iv) sort the slices from the regrouped DICOM files in order of each slice's location along an image patient position of the patient;
(v) merge the sorted slices from each series type into a single three-dimensional (3D) image array and record a position value for each of the slices in the 3D image array;
(vi) form a list of one or more groups comprising the 3D image arrays that are combinable based on sharing the same series type and a location of the patient;
(vii) transform one or more features of the one or more 3D image arrays of each series type in each group to be identical; and
(viii) combine the one or more transformed 3D image arrays of each group using the position value of each slice of the 3D image arrays and a slice spacing.

2. The system of claim 1, wherein the one or more properties related to the one or more series and one or more series types include a weighting, a fat saturation, a component, a resolution, a derived/composed setting, the image patient position, a patient orientation, a patient region, a patient region laterality, or any combination thereof.

3-10. (canceled)

11. The system of claim 2, wherein the processer at (iii) is further configured to save each slice from each DICOM file in an image list for each series type and save the image patient position for each slice in the image list in a corresponding image patient position list for each series type.

12. The system of claim 11, wherein the processor further records for each series type an in-plane pixel spacing, a spacing between the slices, a pixel row size, a pixel column size, and the image patient position.

13. The system of claim 11, wherein the processor in (iv) is further configured to sort the slices from the image list in a descending order of the image patient position from the image patient position list.

14. The system of claim 13, wherein the processor further records a first slice and a last slice of the sorted slices from each image list to define a series slice coverage.

15. The system of claim 11, wherein the processor at step (iv) is further configured to (1) select slices at one or more predefined intervals along the sorted slices for each image list and (2) input each selected slice into (a) a region identification algorithm using artificial intelligence (AI) or image processing to label each selected slice as an identified patient region and (b) a region laterality identification algorithm using AI or image processing to label each selected slice as an identified patient region laterality.

16. The system of claim 15, wherein the selected slices are processed by a Sobel filter before being input into the region identification AI and region laterality identification AI.

17. The system of claim 16, wherein each series is defined by the patient region and patient region laterality based on a majority of output slices from each of the region identification AI and region laterality identification AI.

18. The system of claim 17, wherein the processor further confirms an identical region between identical series types with different components and an identical region laterality between identical series types with different components.

19. The system of claim 11, wherein the processor in (vi) is further configured to order each 3D image array by a descending order of the image patient position.

20. The system of claim 19, wherein the processor in (vii) is further configured to combine the grouped 3D image arrays by transforming the features of each 3D image array in each group to have an identical pixel row size, an identical pixel column size, an identical field of view, an identical maximum in-plane resolution, or an identical slice spacing resolution.

21. The system of claim 20, wherein the processor further records the pixel row size, the pixel column size, the maximum in-plane resolution, the slice spacing resolution, and the FOV for each 3D image.

22. The system of claim 21, wherein the processor transforms each 3D image array in each group by (1) padding any slices within each 3D image array with zeros that have smaller pixel row or pixel column than a maximum pixel row and maximum pixel column of the group, (2) resampling each 3D image array to the maximum in-plane resolution and the slice spacing resolution of the group, (3) rolling the 3D image array using the FOV of the group, or (4) any combination thereof.

23. The system of claim 11, wherein the processor in (viii) is further configured to identify and record any 3D image arrays in each group that overlap with more than one adjacent 3D image arrays in the same group by more than a first slice and a last slice of adjacent 3D image arrays.

24. The system of claim 23, wherein the processor further produces all combinable versions based on the recorded, overlapping 3D image arrays.

25. The system of claim 24, wherein the combinable versions include a version in which all recorded, overlapping 3D image arrays in the group are used, one or more versions in which all but one of the recorded, overlapping 3D image arrays are used, and a version in which none of the recorded, overlapping 3D image arrays are used.

26. The system of claim 25, wherein each version is combined using a spacing between the slices and a range of the slices in each 3D image array between the first slice and the last slice of each adjacent 3D image array in each group.

27. The system of claim 26, wherein an equal amount of slices from each adjacent 3D image array are removed, an equal amount of padding is added to each adjacent 3D image array, or a combination thereof for adjacent 3D image arrays overlapping by more than one slice.

28. The system of claim 27, wherein the processor further determines a correlation coefficient between pixel intensities of neighboring slices between adjacent 3D image arrays in each group.

29. The system of 28, wherein the processor is further configured to select a single version of the combinable versions with a least amount of padding added and a highest minimum correlation coefficient between all adjacent 3D image arrays in each group.

30. The system of claim 2, wherein the processor is further configured to check and correct swapping between a fat component and a water component between two or more combined 3D images of the same series type.

31. The system of claim 1, wherein the processor in (ii) is further configured to identify and exclude DICOM files from (1) a non-MRI modality, (2) DICOM files that are a survey, localizer, scout series, calibration, or summary series, (3) DICOM files that are not an image series, (4) DICOM files where the image series is not 3D, or (4) any combination thereof.

32. A method for preprocessing magnetic resonance imaging (MRI) scans of a patient acquired with a medical imaging system, the method comprising:
(i) accessing the one or more MRI scans, wherein each MRI scan includes one or more Digital Imaging and Communications in Medicine (DICOM) files acquired from a single study instance unique identifier (UID), and wherein each DICOM file includes a two-dimensional (2D) image array slice;
(ii) separating the one or more DICOM files grouped based on one or more series instance UIDs and regrouping the one or more DICOM files into one or more series;
(iii) identifying and recording one or more properties related to the one or more series and group the series based on one or more proprieties into different series types;
(iv) sorting the slices from the regrouped DICOM files in order of each slice's location along an image patient position of the patient;
(v) merging the sorted slices from each series type into a single three-dimensional (3D) image array and recording a position value for each of the slices in the 3D image array;
(vi) forming a list of one or more groups comprising the 3D image arrays that are combinable based on sharing the same series type and a location of the patient;
(vii) transforming one or more features of the one or more 3D image arrays of each series type in each group to be identical; and
(viii) combining the one or more transformed 3D image arrays of each group using the position value of each slice of the 3D image arrays and a slice spacing.

33. The method of claim 32, further comprising checking and correcting swapping between a fat component and a water component between two or more combined 3D images of the same series type.
